(19) United States
(12) Patent Application Publication
(10) Pub. No.: US 2024/0180940 A1
(43) Pub. Date: Jun. 6, 2024

(54) MPLA COMPOSITIONS AND METHODS OF USE

(71) Applicant: Revelation Biosciences, Inc., San Diego, CA (US)

(72) Inventors: James M. Rolke, Tucson, AZ (US); Jackson R. Stephens, San Diego, CA (US); Joseph M. Ninosky, La Jolla, CA (US)

(21) Appl. No.: 18/562,719

(22) PCT Filed: May 26, 2022
(86) PCT No.: PCT/US22/31060

§ 371 Date: Nov. 20, 2023

Related U.S. Application Data
(60) Provisional application No. 63/193,799, filed on May 27, 2021.

(51) Int. Cl. A61K 31/7024; A61K 9/107; A61K 9/16; A61K 47/10

(52) U.S. Cl. CPC A61K 31/7024; A61K 9/1075; A61K 9/1623; A61K 9/1652; A61K 47/10

(57) ABSTRACT

Disclosed are pharmaceutical compositions of monophosphoryl lipid A (MPLA) like compounds, as well as methods of preparing such pharmaceutical compositions, and method of use for such compositions in treating allergic disease.

### RELATED APPLICATIONS

This application claims the benefit of priority to U.S. Provisional Patent Application No. 63/193,799, filed on May 27, 2021, which is hereby incorporated by reference in its entirety.

### BACKGROUND

Allergic disease (“allergies”) is perhaps the most preeminent area of immunology investigation due to prevalence, relevance as a public health issue, and implications to patient outcomes such as burden of disease. Allergens (e.g., pollens, fungi, animals, dust mites, cockroaches, and venom) are non-infectious substances from the environment that affect genetically predisposed individuals. United States population data collected in 2018 estimated that approximately 7.7% and 7.2% of adults and children, respectively, suffered from allergic rhinitis symptoms (hay fever); 9.6% of children suffered from respiratory allergies. Collectively, allergic diseases constitute a spectrum of health severities, making disease management a challenge to surmount.

Allergic reactions begin in susceptible individuals after exposure to a sufficient amount of allergen, also called “antigen” (e.g., grass pollen, dust mites). There are 2 types of clinical distinctions that characterize allergic manifestations in susceptible individuals: IgE mediated (or IgE-dependent) and non-IgE-mediated (or IgE-independent). Allergic rhinitis, asthma, and venom allergies are major IgE-mediated allergy disorders, especially in children (Alvaro-Lozano 2020). Individuals who produce IgE-mediated allergy signs and symptoms have Type I hypersensitivity (1 of 4 types) and are often classified as “atopic” patients due to their genetic predisposition and tendency to develop sensitivities earlier in life.

Allergic mechanisms at the cellular level vary between different antigens. Allergic rhinitis, for example, develops via a process of sensitization, challenge, and elicitation, and encompasses both the innate and adaptive immune systems. Type I hypersensitivity results in activation of a Th2 response toward antigen, resulting in allergen-specific IgE antibody production (Bousquet 2020). Allergen-specific IgE antibodies bind to mast cell and basophil receptors upon subsequent challenge with antigen, resulting in degranulation and release of inflammatory mediators (e.g., histamine, leukotrienes, tryptase). These mediators cause mobilization of basophils, eosinophils, and T lymphocytes to the site of insult, as well as activation of T cells, which compound symptomology and enhance inflammation. Systemic responses, e.g., anaphylaxis, may also occur within minutes or hours post-challenge. Accordingly, there remains a significant need for effective therapies for treating and preventing allergic disease.

Stimulation of TLR4 results in mobilization of exosomes from epithelia which can release antimicrobial peptides and nitric oxide that can destroy invading pathogens/antigens (Nocera 2018). TLR4 stimulation also leads to generation of Type I interferons (IFNs) which block activation of Th2 cellular activity, resulting in reduced IgE secretion and reduction in allergic symptoms (Gonzalez-van Horn 2015). Type I IFN response results in the generation of IP-10 (CXCL10), which is capable of binding to CCR3, the native receptor for eotaxin and is present on cells involved in the allergic response, including eosinophils, basophils, and mast cells (Loetscher 2001). IP-10 binding to CCR3 prevents eotaxin from binding and recruiting immune cells, thereby reducing recruitment of Th2 cells and attenuating the allergic response.

In addition to allergic disease, bacterial pathogens have developed ways to exploit and evade the host's innate immune responses. Stimulating a stronger and more effective innate immune response to these microbes is a recognized mechanism to combat early infection. Strengthening of the innate immune response can be achieved through stimulation of the TLR4 pathway, which in turn upregulates the production of certain protective, proinflammatory chemokines and cytokines, such as interferon (IFN). IFNs interfere with bacteremia and increases bacterial clearance by binding to cell surface receptors to activate the transcription of hundreds of IFN-stimulated genes (ISGs). To date, there are no known regulatory agency-approved innate immunological interventions that can be used prophylactically or therapeutically for bacterial infections.

Formulation of MPLA-like compounds is difficult due the hydrophobic nature of the molecule and the need for the formation of a stable micelle to impart biologic activity. While multiple formulations have been described including oil-in-water emulsions, suspensions, nanoparticulate suspensions, liposomal formulations, and aqueous formulations with a cosurfactant, these all suffer from multiple drawbacks including injection site pain, injection site reaction, lack of bioavailability, inability to be given orally, inability to be given parenterally, lack of stability, and lack of utility due to requirements for specialized equipment at the time of use.

Disclosed herein are compositions of MPLA-like compounds that may advantageously be used for treating and preventing allergic disease.

### SUMMARY

Provided herein are pharmaceutical compositions comprising monophosphoryl lipid A (MPLA) like compounds. MPLA-like compounds refers to any compound with the general structure shown in FIG. 1. The common feature of all MPLA-like compounds is the monophosphorylated disaccharide. The degree of acylation can vary ranging from as few as 4 acyl groups to as many as 9 acyl groups. In addition, the length of each acyl chain (e.g. the number of carbons) can vary from about 8 to about 20 carbons.

MPLA-like compounds may be either synthetic or biologically derived as described (e.g. from hydrolysis of bacterial cell walls). In certain preferred embodiments, the MPLA is selected from phosphorylated hexaacyl disaccharide (PHAD), PHAD-504, 3D-(6-acyl)-PHAD, 3D-PHAD, and any combination thereof. In certain preferred embodiments, the MPLA is PHAD.

In some embodiments of the invention, allergic disease can be treated by administering to a subject an effective amount of an MPLA-like compound. Also provided are methods for delivering an MPLA-like compound to a subject in need thereof. In some embodiments, the MPLA-like compound is delivered locally to mucosal tissue. In some preferred embodiments, the TLR4 agonist is delivered intranasally. In other embodiments the MPLA-like compound is delivered systemically. In some preferred embodiments, the MPLA-like compound is delivered orally, intravenously, subcutaneously, or intramuscularly.

Also provided are methods of preparing pharmaceutical compositions disclosed herein, comprising:
a. dissolving one or more MPLA-like compounds in an organic solvent to form an organic solvent/MPLA solution;
b. combining the organic solvent/MPLA solution with water to form a colloid comprising the one or more MPLAs.

### BRIEF DESCRIPTION OF THE DRAWINGS

FIG. 1 depicts the structure of synthetic phosphorylated, hexaacyl disaccharide (PHAD).

FIG. 2 IP-10 upregulation as a function of dose using representative formulated PHAD. Multiple preparations of PHAD were assessed in an in vitro cell based activity assay using mouse macrophages of the J774 cell line to demonstrate stimulation of TLR4 in response to PHAD as measured by upregulation of IP-10.

FIG. 3 Monophosphorolipid A (MPLA) like compounds can preferentially stimulate TLR4 to activate the TRIF pathway leading to the production of protective cytokines

FIG. 4 The stimulation of TLR4 in response to MPLA-like compounds leads to generation of Type I interferons preferentially through the TRIF pathway (FIG. 3). A negative reciprocal feedback loop has been observed to exist between Type I interferon activity and Th2-biased cellular activity.

FIG. 5 Representative structure of lipopolysaccharide obtained from gram-negative bacteria.

FIG. 6 Results of an ad hoc analysis of IP-10 upregulation in response to composition of PHAD contemplated in the current invention in normal healthy human volunteers.

FIG. 7 depicts the structure of monophosphoryl 3-deacyl lipid A.

FIG. 8 depicts the structure of 3D-(6-acyl) PHAD (or 3,6-acyl PHAD) (FIG. 8).

### DETAILED DESCRIPTION

### Definitions

“About” and “approximately” shall generally mean within an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. “Colloid” as used herein, refers to any liquid or solid composition comprising multimolecular aggregate microstructures having diameters or lengths on the scale of 1 nm to 10 μm. Such microstructures include but are not limited to micelles, liposomes, vesicles, nanoparticles, microparticles, etc. The microstructures may be spherical, oval, oblong, flat, or any other shape.

“Micelles,” as used herein, is an art-recognized term and refers to particles of colloidal dimensions that exist in equilibrium with the molecules or ions in solution from which it is formed. It is an aggregate (or supramolecular assembly) of molecules dispersed in a liquid, forming a colloidal suspension (also known as associated colloidal system). A typical micelle in water forms an aggregate with the hydrophilic “head” regions in contact with surrounding solvent, sequestering the hydrophobic single-tail regions in the micelle center.

“Liposome,” as used herein, is an art-recognized term and refers to a spherical vesicle having at least one lipid bilayer. Liposomes can be prepared by disrupting biological membranes (such as by sonication).

“Vesicle,” as used herein is an art-recognized term and refers to a membranous fluid filled sac surround by a lipid bilayer.

“Nanoparticle,” as used herein, is an art-recognized term and is typically defined as a particle of matter that is between 1 and 100 nanometers (nm) in diameter. The term is sometimes used for larger particles, up to 500 nm.

“Microparticle,” as used herein, is an art-recognized term and is defined to be particles between 1 and 1000 μm in size.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof). Treating a respiratory viral infection may include: alleviation or elimination of symptoms such as runny nose, sneezing, itchy watery eyes, cough, fatigue, headache, sore throat, or congestion.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “patient,” “subject,” or “individual” are used interchangeably and refer to either a human or a non-human animal. These terms include mammals, such as humans, non-human primates, livestock animals (including bovines, porcines, etc.), companion animals (e.g., canines, felines, etc.) and rodents (e.g., mice and rats). In some embodiments, the subject is a human.

The phrase “pharmaceutically acceptable excipient” as used herein means a pharmaceutically acceptable material, composition or vehicle, such as a liquid or solid filler, diluent, lubricant, binder, carrier, humectant, disintegrant, solvent or encapsulating material, that one skilled in the art would consider suitable for rendering a pharmaceutical formulation suitable for administration to a subject. Each excipient must be “acceptable” in the sense of being compatible with the other ingredients of the formulation, as well as “pharmaceutically acceptable” as defined above. Examples of materials which can serve as pharmaceutically acceptable excipients include but are not limited to: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; silica, waxes; oils, such as corn oil and sesame oil; glycols, such as propylene glycol and glycerin; polyols, such as sorbitol, mannitol and polyethylene glycol; esters, such as ethyl oleate and ethyl laurate; agar; buffering agents; alginic acid; pyrogen-free water; isotonic saline; Ringer's solution; and other non-toxic compatible substances routinely employed in pharmaceutical formulations.

A “therapeutically effective amount” or a “therapeutically effective dose” of a drug or agent is an amount of a drug or an agent that, when administered to a subject, will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated.

### Allergies and the Innate Immune System

While not being bound by theory, it is believed that compositions comprising an MPLA compound according to the present invention are capable of stimulating the innate immune system to produce certain protective cytokines, including interferons (IFNs) and interleukins (ILs). MPLA upregulates protective cytokines via interaction with Toll-like receptor 4 (TLR4), which upon stimulation activates 2 signal transduction pathways: one mediated by myeloid differentiation factor 88 (MyD88); and another mediated by toll-IL-1 receptor domain containing adaptor inducing IFN-β (TRIF). MPLA upregulates specific cytokines, including IFN-γ (Type II IFN), Type I IFNs (IFN-α and IFN-β), IP-10, IL-17, and IL-8 which are secreted from specific immune cells.

In addition, the upregulation of Type I IFNs have demonstrated specific antibacterial capability against gram negative bacteria. Although IFNs have been used in previous treatments or combination treatments, the timing and method of administration of IFN is vital to its effectiveness against infection. In contrast to exogenous interferon, compositions comprising an MPLA-like compound in accordance with the invention offer a number of advantages, including the elicitation of the innate immune response, notably, the increased expression of endogenous Type I interferons.

Synthetic and bacterially-derived MPLA-like compounds are recognized by toll-like receptor 4 (TLR4). Interaction with TLR4 stimulates the production of protective cytokines including interferons (IFNs) (e.g., IFN-α, IFN-γ), IP-10, IL-8, IL-17, and TNF-α, or combinations thereof. Interferons are a type of cytokine associated with the innate immune system and are released by sentinel cells (e.g., macrophages, dendritic cells) when TLRs on sentinel cells come in contact with PAMPs. Interferons interfere with bacteremia, increase bacterial clearance, and are the body's first line of defense against bacterial infections, binding to cell surface receptors to activate the transcription of hundreds of genes.

Robust upregulation of IP-10, as a result of TLR4 stimulation by MPLA-like compounds, has been demonstrated in vitro in mouse macrophages (FIG. 2). Interferon-□ is required for production on IP-10, therefore IP-10 is considered a surrogate marker for Type I interferon activity.

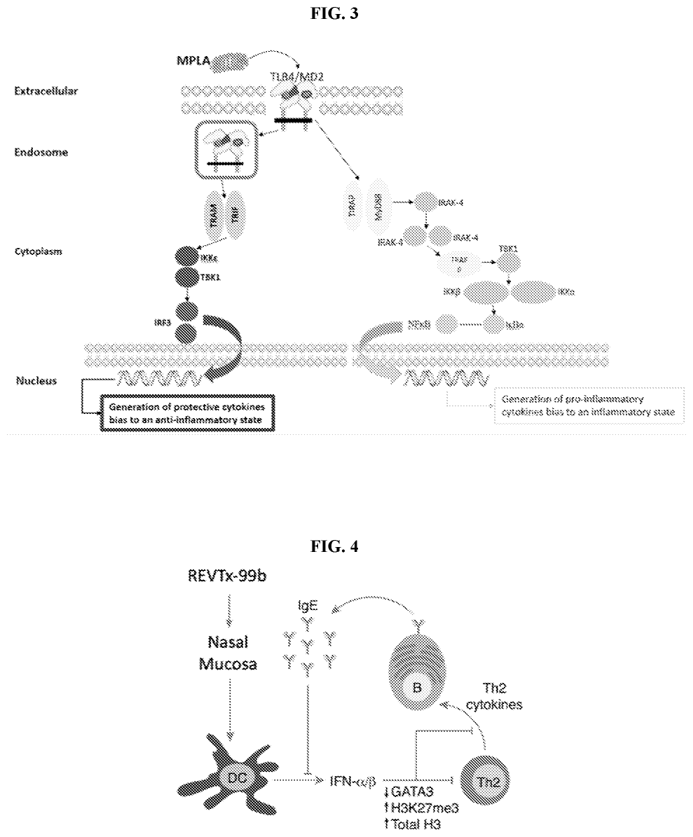

The stimulation of TLR4 in response to MPLA-like compounds leads to generation of Type I interferons preferentially through the TRIF pathway (FIG. 3). A negative reciprocal feedback loop has been observed to exist between Type I interferon activity and Th2-biased cellular activity (Gonzalez-van Horn 2015). This relationship is illustrated in FIG. 4, which details Type I interferon production blocking activation of Th2 cellular activity at the molecular level (suppression of GATA3 expression, preventing access to transcription mediated expression for H3K27me3 and Total H3), which prevents generation of Th2 cytokines (IL-4, IL-5, and IL-13), reducing or eliminating secretion of IgE, therefore reducing or preventing allergic symptoms. It has been demonstrated that Type I interferons reduce or prevent Th2-biased cellular activity, which facilitates the allergic response, destabilizing establishment of Th2-biased cellular activity.

REVTx-99 is also capable of reducing eotaxin activation through indirect competition for the native receptor, CCR3, as IP-10 is capable of binding to CCR3. In addition to this activity, REVTx-99 is further capable of reducing allergic symptoms via TLR4 mediated recruitment of exosomes from stimulated epithelial cells. In the anterior portion of the nose, these exosomes release antimicrobial peptides and nitric oxide into nasal mucus that can destroy foreign invaders.

Early treatment with MPLA may augment interferon in patients with respiratory bacterial infections, inhibit bacteremia, avoid the accumulation of dangerously high bacterial loads, signal the immune system, and suppress cytokine storm. MPLA may be able to treat or prevent allergic disease and the associated congestion-causing inflammation by reducing or blocking the signaling and recruitment of cytotoxic immune cells.

### Dosing and Administration

The MPLA-like compound can be administered systemically or locally (e.g. directly to the nasal mucosa).

The current invention contemplates using MPLA-like compounds as treatment for allergic rhinitis. MPLA upregulates protective cytokines via interaction with Toll-like receptor 4 (TLR4), which upon stimulation activates two signal transduction pathways: one mediated by myeloid differentiation factor 88 (MyD88); and another mediated by toll-IL-1 receptor domain containing adaptor inducing IFN-β (TRIF). MPLA upregulates specific cytokines, including IFN-γ (Type II IFN), Type I IFNs (IFN-α and IFN-β), IP-10, IL-17, and IL-8 which are secreted from specific immune boosting cells generated by the spleen.

In some embodiments, the TLR4 agonists described herein selectively activate the TRIF pathway in favor of the MyD88 pathway. In certain embodiments, the TLR4 agonist has an EC₅₀ for activating the TRIF pathway and its EC₅₀ for activating the MyD88 pathway have a ratio of about 1.1:1 to greater than 100,000:1. In some embodiments, the ratio of these EC₅₀s is greater than about 1.1:1, greater than about 1.5:1, greater than about 2:1, greater than about 5:1, greater than about 10:1, greater than about 100:1, greater than about 200:1, greater than about 500:1, greater than about 1000:1, greater than about 5,000:1, greater than about 10,000:1, or greater than about 100,000:1. In some embodiments, the ratio of these EC₅₀s lies in a range of about 1:1 to about 100,000:1, about 1.1:1 to about 50,000:1, about 1.1:1 to about 10,000:1, about 1.1:1 to about 1,000:1, about 1.1:1 to about 100:1, about 1.1:1 to about 10:1, or about 1.1:1 to about 5:1.

In some embodiments, allergic rhinitis can be treated by administering to a subject an effective amount of a TLR4 agonist. In some embodiments, the TLR4 agonist is an MPLA-like compound. In some preferred embodiments the MPLA-like compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

In some embodiments of the invention, allergic disease, including allergic rhinitis, rhinosinusitis, chronic nasal congestion, asthma, atopic eczema, drug allergies, and skin allergies can be treated by administering to a subject an effective amount of a TLR4 agonist that preferentially stimulates the TRIF pathway. In some preferred embodiments, allergic disease can be treated by administering to a subject an effective amount of an MPLA-like compound. In certain preferred embodiments the allergic disease is allergic rhinitis. In other preferred embodiments the allergic disease is rhinosinusitis, chronic nasal congestion, asthma, atopic eczema, drug allergies, and skin allergies.

In some preferred embodiments the MPLA-like compounds are delivered intranasally as a dry powder. In other preferred embodiments, the MPLA-like compounds are delivered intranasally as an aqueous colloid. In certain preferred embodiments, the MPLA-like compounds are delivered intranasally as an aqueous colloid comprising an MPLA-like compound and one or more sugars.

### MPLA-Like Compounds

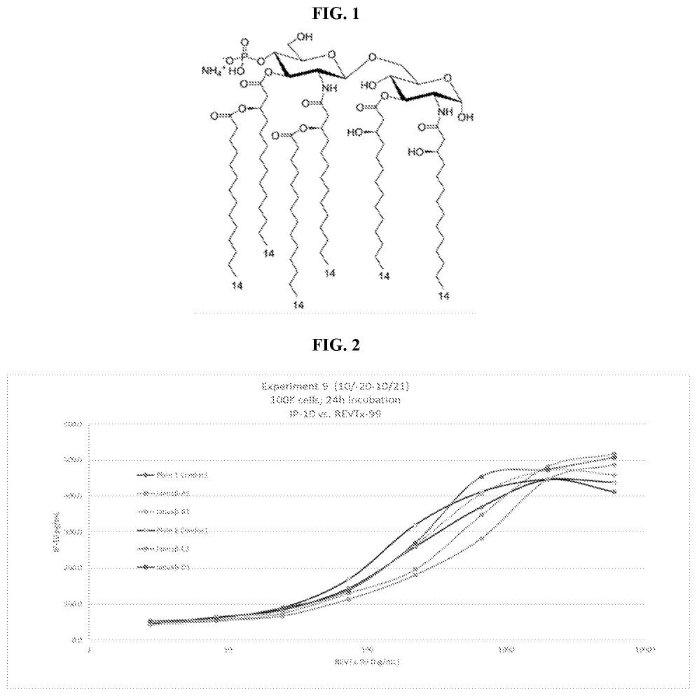

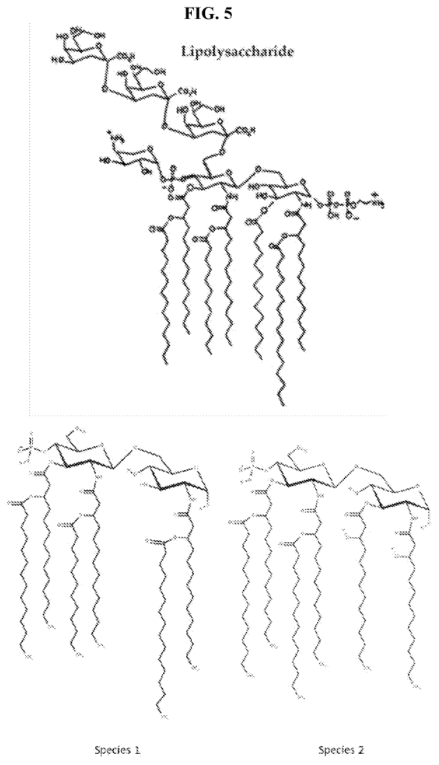

The pharmaceutical compositions of the present disclosure comprise a monophosphoryl lipid A (MPLA) like compound. MPLA was originally isolated from lipopolysaccharide obtained from gram-negative bacterial cell walls (FIG. 5). Bacterially derived MPLA is typically a mixture of several different species. FIG. 1 shows one of the predominant species of bacterially derived MPLA. As an example, MPLA may be derived from Salmonella minnesota R595 lipopolysaccharides. As will be understood, MPLA may also be derived from other Salmonella species. The bacterial LPS may be processed via sequential acid and alkaline hydrolysis steps to remove polysaccharide side chains, phosphate groups, and to partially remove a portion of the acetyl side groups. The crude MPLA may then be purified. The final MPLA product is a mixture of heptaacyl-, hexaacyl-, and pentaacyl-monophosphorylated glucosamine disaccharide linked βlà6. Diaacetyl, triaacetyl, and tetraacetyl, if present, are considered impurities. The acylated lipids vary and include lauroyl, myristoyl, and palmitoyl. While the relative ratio of each species can vary from batch to batch, the predominant species produced are the hexaacylated disaccharide products.

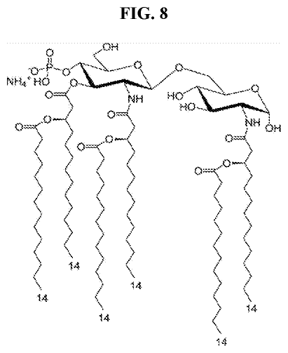

The major species found in bacterially-derived MPLA have been chemically synthesized and have comparable immunostimulatory properties to the bacterially-derived material. Examples of synthetic MPLA-like compounds suitable for use in the present invention include phosphorylated hexaacyl disaccharide (PHAD®) (also known as glucopyranosyl lipid A, or GLA) (FIG. 1), 3D-PHAD (or 3-acyl-PHAD) (also known as monophosphoryl 3-deacyl lipid A) (FIG. 7), and 3D-(6-acyl) PHAD (or 3,6-acyl PHAD) (FIG. 8). Synthetic variations of MPLA that are also suitable and within the scope of the invention include those wherein the fatty acid chain length varies between 10-20 carbons and those wherein the degree of acylation is penta-, hexa-, or hepta-.

PHAD is chemically equivalent to a major component of bacterially-derived MPLA. PHAD is also equivalent to bacterially-derived MPLA in biologic effect.

### Dosage Forms and Routes of Administration

MPLA-like compounds can be administered by several different routes. The choice of the route is dependent on multiple factors including the need (or not) for systemic exposure, the desire for the MPLA-like compound to reach a particular organ quickly, patient tolerability, and compliance.

Methods of systemic delivery include those methods known in the art that provide delivery of the active molecule (e.g. the drug) to the circulatory system with distribution throughout the body. Systemic delivery methods include intramuscular, intravenous, subcutaneous, intraperitoneal, sublingual, and oral. As will be understood, any method of systemic delivery is suitable for use with the invention. Particularly suitable methods of systemic delivery include oral, intramuscular, and intravenous delivery.

In some embodiments, it may desirable to only have the drug interact with the mucosal tissue and for there to be no or minimal systemic exposure. Methods for mucosal delivery include those methods known in the art that provide delivery of the active molecule to mucous membranes. Mucosal delivery methods include intranasal, intrabuccal, sublingual, and oral. Particularly suitable methods for mucosal delivery include intranasal delivery.

In these embodiments, the composition comprising the MPLA-like compound may be formulated to be delivered to the nasal passages or nasal vestibule of the subject as droplets, an aerosol, micelles in solution, lipid or liquid nanospheres, liposomes, lipid or liquid microspheres, a solution spray, or a powder. The composition can be administered by direct application to the nasal passages or may be atomized or nebulized for inhalation through the nose or mouth.

In some embodiments, the method comprises administering a nasal spray, medicated nasal swab, medicated wipe, nasal drops, or aerosol to the subject's nasal passages or nasal vestibule. To this end, viscosity modifying agents that may be deployed to optimize the product for the application format may include cetyl alcohol, stearyl alcohol, carnauba wax, stearic acid, xanthan gum, magnesium aluminum silicate, gelatin, carbomer, poloxamers, PEGs, waxes, starches, castor oil derivatives, fatty acids, fatty alcohols, and lecithin.

In some embodiments, the compositions of the present invention can be delivered using a small needle-free nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single-dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediately prior to use and squeezed or squirted into the nasal passages. In some embodiments, the nasal spray or drops may be accomplished by time of use reconstitution of the product powder with an aqueous vehicle immediately prior to administration. In some embodiments, the nasal spray or drops may be accomplished by reconstitution of the solid drug product powder contained in a suitable delivery device using an aqueous vehicle in some time period in which the drug product is deemed stable in solution format prior to patient administration.

In certain embodiments, the compositions are suitable for parenteral administration to a mammal, most preferably by injection or intravenous infusion, and in some embodiments the compositions may comprise one or more pharmaceutically acceptable excipients. Suitable excipients include pharmaceutically acceptable buffers, stabilizers, local anesthetics, and the like. The composition may be adapted for direct injection or intravenous infusion, or for addition to an intravenous drip solution for gradual infusion, through appropriate use of excipients and packaging and delivery means well-known in the art.

In other embodiments, the invention provides a pharmaceutical package, comprising a vial or ampoule containing an MPLA-like compound in the form of a reconstitutable powder or a solution suitable for injection or infusion, together with instructions for administering the composition to a patient in need thereof. Instructions include but are not limited to written and/or pictorial descriptions of: the active ingredient, directions for diluting the composition to a concentration suitable for administration, suitable indications, suitable dosage regimens, contraindications, drug interactions, and any adverse side-effects noted in the course of clinical trials.

In alternative embodiments, the pharmaceutical package may comprise a plastic bag containing from 100 ml to 2 L of a pharmaceutical composition of the invention, in the form of a solution suitable for intravenous administration, together with instructions as described above.

In alternative embodiments, a pharmaceutical composition of the invention may be in a form adapted for oral dosage, such as for example a syrup or palatable solution; a form adapted for topical application, such as for example a cream or ointment; or a form adapted for administration by inhalation, such as for example a microcrystalline powder or a solution suitable for nebulization. Methods and means for formulating pharmaceutical ingredients for alternative routes of administration are well-known in the art, and it is to be expected that those skilled in the relevant arts can adapt these known methods to the MPLA-like compounds and formulations described in the present invention.

The present invention provides pharmaceutically acceptable compositions comprising a therapeutically effective amount of one or more MPLA-like compounds, formulated together with one or more pharmaceutically acceptable excipients. The pharmaceutical compositions of the present invention may be formulated for administration in solid or liquid form, including forms adapted for oral administration, for example, aqueous or non-aqueous solutions or suspensions, tablets, powders, and granules; administration by inhalation, for example, aerosols, solutions for nebulization, or dry powders; parenteral administration, for example sterile solutions or suspensions; topical application, for example lotions, creams, ointments or sprays; ophthalmic administration; or intravaginal or intrarectal administration, for example pessaries, suppositories, creams or foams. Preferably, the pharmaceutical preparation is adapted for parenteral administration, more preferably it is a non-pyrogenic solution adapted for intravenous administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention may optionally be scored or prepared with coatings and shells, such as enteric coatings and other coatings well known in the pharmaceutical-formulating art. They may also be formulated so as to provide slow or controlled release of the modified therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally in a delayed manner. Examples of embedding compositions that can be used include polymeric substances and waxes. The MPLA-like compound can also be in micro-encapsulated form, if appropriate, with one or more of the above-described excipients.

Liquid dosage forms for oral administration of MPLA-like compounds include pharmaceutically acceptable emulsions, microemulsions, solutions, suspensions, syrups, and elixirs. In addition to the MPLA-like compound, the liquid dosage forms may contain inert diluents commonly used in the art, such as, for example, water or other solvents, solubilizing agents, and emulsifiers.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In dry powder formulations adapted for inhalation, the particle size of the particulate medicament should be such as to permit inhalation of substantially all of the medicament into the lungs upon administration of the aerosol formulation and will thus desirably be less than 20 microns, preferably in the range 1 to 10 microns, more preferably 1 to 5 microns. The particle size of the medicament may be reduced by conventional means, for example by milling or micronization. The aerosol formulation preferably contains 0.5-30% w/w of an MPLA-like compound relative to the total weight of the formulation.

The propellant may optionally contain an adjuvant having a higher polarity and/or a higher boiling point than the propellant. Polar adjuvants which may be used include (e.g. C2-6) aliphatic alcohols and polyols such as ethanol, isopropanol and propylene glycol, preferably ethanol. In general, only small quantities of polar adjuvants (e.g. 0.05-3.0% w/w) may be required to improve the stability of the dispersion. However, the formulations of the invention are preferably substantially free of polar adjuvants, especially ethanol. Suitable propellants include trichlorofluoromethane (propellant 11), dichlorodifluoromethane (propellant 12), dichlorotetrafluoroethane (propellant 114), tetrafluoroethane (propellant 134a) and 1,1-difluoroethane (propellant 152a), saturated hydrocarbons such as propane, n-butane, isobutane, pentane and isopentane, and alkyl ethers such as dimethyl ether. In general, up to 50% w/w of the propellant may comprise a volatile adjuvant, for example 1 to 30% w/w of a volatile saturated C1-C6 hydrocarbon.

The aerosol formulations according to the invention may optionally comprise one or more surfactants that are physiologically acceptable upon administration by inhalation.

For administration by inhalation, the drug is suitably inhaled from a nebulizer, from a pressurized metered dose inhaler or as a dry powder from a dry powder inhaler optionally using gelatin, plastic or other capsules, cartridges, blister packs and/or strips.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. Typically, administration will range from one or to four or more times daily.

For use in dry powder inhalers, the active ingredient can be modified by spray drying or compression to form a powder with suitable flow properties. More commonly a diluent or carrier is added which is generally non-toxic and inert to the medicament. Examples of such carriers are polysaccharides e.g. starch and cellulose, dextran, lactose, glucose, mannitol, and trehalose. The carrier can be further modified by the addition of surface modifiers, pretreatment to form low rugosity particles, addition of glidants, and flavor masking or modifying agents.

Pharmaceutical compositions of this invention suitable for parenteral administration comprise an MPLA-like compound in combination with one or more pharmaceutically acceptable sterile isotonic aqueous or non-aqueous solutions, or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Examples of pharmaceutically acceptable antioxidants include but are not limited to ascorbic acid, cysteine hydrochloride, sodium metabisulfite, sodium sulfite, ascorbyl palmitate, butylated hydroxyanisole (BHA), butylated hydroxytoluene (BHT), propyl gallate, alpha-tocopherol, and chelating agents such as citric acid, ethylenediamine tetraacetic acid (EDTA), sorbitol, tartaric acid, phosphoric acid, and the like.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes, vesicles, or microemulsions that are compatible with body tissue.

Dosage forms for the topical or transdermal administration of a compound of this invention include powders, sprays, ointments, pastes, creams, lotions, gels, solutions, patches and inhalants. The MPLA-like compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Ophthalmic formulations, eye ointments, powders, solutions and the like, are also contemplated as being within the scope of this invention.

Formulations of the present invention which are suitable for vaginal administration include pessaries, tampons, creams, gels, pastes, foams or spray formulations, containing such carriers as are known in the art to be appropriate. Such formulations may be prepared, for example, by mixing one or more MPLA-like compounds with one or more suitable nonirritating excipients comprising, for example, cocoa butter, polyethylene glycol, or a suppository wax, which is solid at room temperature but liquid at body temperature and, therefore, will melt in the rectum or vaginal cavity and release the MPLA-like compound.

### Therapeutic Doses and Regimens

In certain embodiments, an MPLA-like compound is administered in a total dose between 0.001 to 10 milligrams. In preferred embodiments, the total dose administered is between 50 and 1000 micrograms. In particularly preferred embodiments, the total dose is about 100 to 500 micrograms. In certain embodiments, MPLA is administered in a total dose between 0.1 to 200 micrograms. The total dose given may be administered intranasally, and may be divided in equal or unequal parts between both nostrils. In preferred embodiments, the total dose administered is between 10 and 100 micrograms. In particularly preferred embodiments, the total dose is about 50 to 100 micrograms.

In other embodiments of the invention, an MPLA-like compound is given as a single dose. In other embodiments, MPLA-like compound is given multiple times. In the case of multiple doses, MPLA-like compounds may be given daily, bi-weekly, weekly, or monthly. The exact frequency of dosing and the dose required at each interval will depend on multiple factors including the type of allergic condition being treated, the rate of allergic relapse, and patient tolerability. Early in the treatment phase, the dose and/or dosing frequency may be greater with a gradual decrease in both dose and/or dosing frequency as the allergic response diminishes to maintenance of a steady state (lack of allergic response) In some embodiments of the invention, the MPLA-like compound is administered prior to, simultaneously, or after the onset of allergic symptoms.

### Compositions and Methods of Preparation

In certain embodiments, the pharmaceutical composition is an aqueous composition. In some such embodiments, the pharmaceutical compositions may contain an organic solvent. In certain embodiments, the organic solvent may comprise up to 15% of the total end volume of the administered drug product solution. In preferred embodiments, the organic solvent may comprise up to 5% of the total end volume of the administered drug product solution.

In certain embodiments, the organic solvent is miscible with water, such as an organic solvent selected from an alcohol, glycerin, low molecular weight polyethylene glycol, and low molecular weight poloxamers. In certain preferred embodiments, the organic solvent is an alcohol, e.g., methanol, ethanol, isopropanol, t-butanol, or preferably ethanol.

In certain embodiments, the composition further comprises a sugar. In some embodiments, the sugar is chosen from a monosaccharide, a disaccharide, a trisaccharide, a linear oligosaccharide, a branched oligosaccharide, a cyclic oligosaccharide, a linear polysaccharide, a branched polysaccharide, or any combination thereof.

In some embodiments, the monosaccharide is selected from glucose, dextrose, fructose, galactose, xylose, ribose, and any combination thereof.

In some embodiments, the disaccharide is selected from trehalose, sucrose, maltose, lactose, and any combination thereof.

In some embodiments, the trisaccharide is selected from nigerotriose, maltotriose, melezitose, maltotriulose, raffinose, kestose. and any combination thereof.

In some embodiments, the linear or branched oligosaccharide is selected from nigerotetraose, maltotetraose, lychnose, nystose, sesamose, stachyose.

In some embodiments, the cyclic oligosaccharide is selected from alpha-cyclodextrin, beta-cyclodextrin, or gamma-cyclodextrin.

In some embodiments, the linear or branched polysaccharide is selected from starch, glucan, chitosan, pectin, carboxymethyl cellulose, glycosylaminoglycans, hyaluronic acid, cellulose derivatives, hydroxypropylmethylcellulose (HPMC), dextran, and any combination thereof.

In some preferred embodiments, the sugar comprises trehalose, beta-cyclodextrin, or both.

In certain embodiments, the pharmaceutical compositions further comprise one or more surfactants. In certain embodiments, the one or more surfactants are selected from carboxymethyl cellulose, dodecyltrimethylammonium bromide (DTAB), n-dodecyl octa(ethylene oxide) (C12E8), n-dodecyl tetra (ethylene oxide) (C12E4), dioctanoyl phosphatidylcholine (C8-lecithin), Polyoxyl 35 castor oil, Cremophor EL (CrEL), Octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), and sodium dodecyl sulfate (SDS). In certain preferred embodiments, the one or more surfactants is carboxymethyl cellulose.

In certain embodiments, the pharmaceutical compositions may contain excipients, additives, bulking agents, and mucoadhesive agents. These may include mannitol, trehalose, cyclodextrin, and hydroxypropyl methylcellulose (HPMC). In preferred embodiments, the excipients HP-β-Cyclodextrin and trehalose are employed.

In certain embodiments, the pharmaceutical compositions further comprise a phospholipid selected from phosphatidic acid (PA), phosphatidylcholine (PC), phosphatidylglycerol (PG), phophatidylethanolamine (PE), phophatidylinositol (PI), phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (DMPG), dioleoylphosphatidylglycerol (DOPG), dipalmitoylphosphatidylglycerol (DPPG), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (“DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), di decanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinolcoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In certain embodiments, the pharmaceutical composition is free or substantially free of salt (e.g., NaCl).

In certain embodiments, the pharmaceutical composition has an MPLA concentration of about 1 μg/mL to about 10,000 μg/mL. In certain embodiments, the pharmaceutical composition has an MPLA concentration of about 50 μg/mL to about 200 μg/mL. In certain embodiments, the pharmaceutical composition has an MPLA concentration of about 125 μg/mL. In certain embodiments, the pharmaceutical composition has an MPLA concentration of about 250 g/mL.

In certain embodiments, the pharmaceutical compositions further comprise a stabilizer or multiple stabilizers. In certain preferred embodiments, the stabilizer is trehalose. In other preferred embodiments, the stabilizer is HP-β-Cyclodextrin. In some most preferred embodiments, both HP-β-Cyclodextrin and trehalose are used as stabilizers.

In certain embodiments, the pharmaceutical composition is a dry powder.

In certain embodiments, the colloidal or micellar solution comprises particles having an average diameter of about 1 nm to about 1000 nm. In certain preferred embodiments, the particles have an average diameter of about 50 nm to about 500 nm. In other more preferred embodiments, the particles have an average diameter of about 100 nm to about 500 nm. In other embodiments, the particles have an average diameter of about 500 nm to about 1000 nm. In certain embodiments, the particles have an average diameter of about 10 nm to about 200 nm. In preferred embodiments, the particles have an average diameter of about 10 nm to about 100 nm. In certain embodiments, the particles have an average diameter of less than 50 nm. In certain embodiments, the particles have an average diameter of less than 100 nm. In certain embodiments, the particles have an average diameter of less than 150 nm. In certain embodiments, the particles have an average diameter of less than 200 nm. In certain embodiments, the particles have an average diameter of less than 250 nm. In certain embodiments, the particles have an average diameter of less than 500 nm.

In certain embodiments, the pharmaceutical compositions further comprise a mucoadhesive agent. In certain embodiments, the mucoadhesive agent is selected from cellulose derivatives, polyacrylates, a starch, chitosan, glycosylaminoglycans, hyaluronic acid, cellulose derivatives, polyacrylates, and any combination thereof.

In certain preferred embodiments, the pharmaceutical compositions further comprise a pH modifier, an emulsifier, a pH buffer, a tonicity modifier, a stabilizer, a preservative, a surfactant, a bulking agent, a flavorant, or any combination thereof.

In certain embodiments, the bulking agent is selected from mannitol, trehalose, chitosan, HP-β-Cyclodextrin, hydroxypropylmethylcellulose (HPMC), dextran, starch (such as pea starch), and sucrose.

In certain embodiments, the colloid comprises micelles. In certain embodiments, the colloidal suspension comprises liposomes. In certain embodiments, the colloidal suspension comprises nanoparticles. In certain embodiments, the colloidal suspension comprises microparticles.

In certain embodiments, provided are methods of preparing a pharmaceutical composition disclosed herein, comprising:
a. dissolving one or more MPLAs in an organic solvent to form an organic solvent/MPLA solution;
b. combining the organic solvent/MPLA solution with water to form a colloidal formulation comprising the one or more MPLAs.

In certain embodiments, step (a) or (b) is performed under sonication.

In certain embodiments, the organic solvent and the water are present in a volume to volume ratio of about 1:1500 to about 1:50. In certain embodiments, the organic solvent and water are present in a volume to volume ratio of about 1:1000 to about 1:100. In certain embodiments, the organic solvent and water are present in a volume to volume ratio of about 1:800.

In certain embodiments, one or more surfactants are added in step (a) or (b). In some preferred embodiments, the surfactant is carboxymethyl cellulose. In another embodiments, one or more phospholipids is added in step (a) or (b). In certain embodiments, the mixture in step (b) is lyophilized. In other embodiments, the mixture in step (b) is spray dried. In some preferred embodiments, the mixture in step (b) is stabilized with trehalose. In certain embodiments, step (a) or step (b) is performed at an elevated temperature. In certain embodiments, the elevated temperature is about 30° C. to about 50° C. In certain preferred embodiments, the elevated temperature is about 40° C. In certain embodiments, the mixture in step (b) has an MPLA concentration of about 125 μg/mL.

In certain embodiments, the mixture in step (b) has an MPLA concentration ranging from about 1 μg/mL to about 1000 μg/mL; about 20 μg/mL to about 500 μg/mL, about 100 μg/mL to about 300 μg/mL, about 250 μg/mL, or about 125 μg/mL.

MPLA-like compoundMPLA-like compoundMPLA-like compoundMPLA-like compoundMPLA-like compoundMPLA-like compoundMPLA-like compound In certain embodiments, the spray-dried powder has an MPLA concentration of 0.25 to 10% w/w MPLA/solids in the final powder. In certain preferred embodiments, the MPLA concentration in the final powder is 0.25 to 2% w/w MPLA/solids.

In certain embodiments, the spray dried powder is reconstituted with water prior to administration to the patient.

In some embodiments, the MPLA-like compound is administered in a composition comprising one or more pharmaceutically acceptable excipients. The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

For example, the composition comprising the MPLA-like compound can be formulated for intranasal delivery as a dry powder, as an aqueous solution, an aqueous suspension, a colloid, a water-in-oil emulsion, a micellar formulation, or as a liposomal formulation.

In some embodiments, the MPLA composition comprises micelles of the MPLA-like compound. While not being bound by theory, it is believed that micelles enhance the activity of the MPLA. The size of the micelles, in some embodiments, is about 50 nm to about 1000 nm. The size of micelles may be measured by various techniques, including dynamic light scattering (DLS), scanning electron microscopy (SEM), and transmission electron microscopy (TEM). Accordingly, in some embodiments, the size of the micelles is about 50 nm to about 1000 nm as measured by DLS.

In certain preferred embodiments, the composition further comprises an organic solvent, such as an alcohol, glycerin, low molecular weight polyethylene glycol, a poloxamer, or any combination thereof. In some embodiments, the organic solvent is water miscible. In some embodiments, the organic solvent is an alcohol, such as methanol, ethanol, isopropanol, or t-butanol, preferably ethanol.

In some embodiments, the composition further comprises a fatty acid salt, fatty acids, a phospholipid, or any combination thereof.

In some embodiments, the composition comprises a phospholipid or a mixture of phospholipids. Examples of phospholipids include, but are not limited to, phosphatidic acid (PA), phosphatidylcholine (PC), phosphatidylglycerol (PG), phophatidylethanolamine (PE), phophatidylinositol (PI), and phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (DMPG), dioleoylphosphatidylglycerol (DOPG), dipalmitoylphosphatidylglycerol (DPPG), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), didecanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In certain embodiments, the phospholipid is DPPC, DOPC, cholesterol, or a mixture thereof.

In certain embodiments, compositions comprising MPLA may contain pH modifiers, pH buffers, oils/emulsifiers (e.g., squalene), tonicity modifiers, stabilizers, preservatives, detergents, flavorants, bulking agents, or secondary immunostimulatory agents. In some embodiments, the composition is a dry powder comprising a bulking agent.

Secondary immunostimulatory agents include, e.g., gonadocorticoids, deoxycholic acid, vitamin D, and beta-glucans. Suitable buffers include sodium chloride-based or potassium chloride-based solutions such as phosphate buffered saline, potassium buffered saline, calcium chloride, sodium lactate, sodium bicarbonate, or borate buffered saline. In some embodiments, the buffer may contain salts, detergents, or carbohydrates which preserve the MPLA upon drying and aid in resolubilizing the MPLA upon encounter with a liquid. Suitable carbohydrates include trehalose, sucrose, glucose, and mannose.

In some embodiments, the composition further comprises a mucoadhesive. Suitable mucoadhesives include: glycosylaminoglycans (GAGS) including chondroitin sulfate, chitosan, hyaluronic acid, cellulose derivatives, HP-β-Cyclodextrin, polyacrylates, starch, HPMC and any combination thereof.

In some embodiments, the mucoadhesive is present in the composition in an amount ranging from about 0.1 to about 50% by weight, about 25% to about 50% by weight, or about 49% by weight.

In some embodiments, the composition further comprises a sugar. Examples of sugars that may be used in the methods provided herein include, but are not limited to, sucrose, glucose, fructose, lactose, maltose, mannose, galactose, trehalose, and combinations thereof. In certain embodiments, the sugar content is about 49% by weight.

In some embodiments, the composition is an aqueous liquid. In such embodiments, the concentration of the MPLA-like compound in the composition may be about 1 μg/mL to about 1000 μg/mL, about 20 μg/mL to about 500 μg/mL, about 100 μg/mL to about 300 μg/mL, or about 250 μg/mL.

In certain embodiments, the formulation may contain ionic or nonionic surfactants. Suitable surfactants include poloxamer 407, poloxamer 181, dodecyltrimethylammonium bromide (DTAB), n-dodecyl octaethylene oxide (C12E8), n-dodecyl tetraethylene oxide (C12E4) and dioctanoyl phosphatidylcholine (C8-lecithin), polyoxyl 35 castor oil, cremophor EL (CrEL), octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), and sodium dodecyl sulfate (SDS) and any combination thereof.

In some embodiments, the MPLA formulation has a pH between 4 and 9. In certain preferred embodiments, the pH is between 5 and 8.

In certain embodiments, the formulations may be free of or substantially free of phospholipids, surfactants, salt (e.g., NaCl), and/or buffers. Substantially free means that the substance in question makes up less than 0.5%, less than 0.25%, less than 0.1%, less than 0.05%, less than 0.01%, or less than 0.005% of the composition by weight.

In some embodiments of the invention, the composition comprises an MPLA-like compound at a concentration between 1 and 8000 μg/mL. In certain preferred embodiments, the MPLA is present at a concentration between 20 and 500 μg/mL. In certain more preferred embodiments, the concentration is between 100 and 300 μg/mL. In certain embodiments, the concentration is 250 μg/mL, and in still other embodiments, the concentration is 125 μg/mL.

In some embodiments of the invention, the solution is formulated such that a surfactant is included at a concentration between 1 and 40% w/w, which can enhance the absorption of the drug upon administration by preventing degradation/metabolism, enhancing barrier permeability via transient opening of tight junctions, disruption of lipid bilayer packing/complexation/carrier/ion pairing and enhancing resident time/slowing down mucociliary clearance. In certain preferred embodiments, the surfactant concentration is between 1 and 25% w/w. In certain most preferred embodiments, the surfactant concentration is 15% w/w. Surfactants of interest include, but are not limited to, dipalmitoyl phosphatidyl choline, soybean lecithin, phosphatidylcholine, sodium taurocholate, sodium deoxycholate sodium, glycodeoxycholate, palmitic acid, stearic acid, and oleic acid.

In some embodiments of the invention, the composition comprises a mucoadhesive at a concentration between 0.1 to 50% w/w, which can enhance the absorption of the drug upon administration by enhancing resident time and/or slowing down mucociliary clearance. In certain preferred embodiments, the mucoadhesive is included at a concentration between 40 to 50% w/w. In certain most preferred embodiments, the mucoadhesive is included at a concentration of 49% w/w. Mucoadhesives of interest include, but are not limited to, cellulose derivatives, HP-β-Cyclodextrin polyacrylates, starch, and chitosan.

### Combination Therapies

There are a number of medications currently available for allergies, including antihistamines, decongestants, and steroidal sprays. Over the counter oral antihistamines include Benadryl (diphenhydramine), Claritin (loratadine), Allegra (fexofenadine), and Zyrtec (cetirizine), and nasal sprays such as Nasahist B (brompheniramine). Prescription oral antihistamines include Clarinex (desloratadine), and nasal sprays such as Astelin (azelastine nasal). Some of the decongestants available for treatment include Sudafed (pseudophedrine), Neo-Synephrine (phenylephrine), and Afrin (oxymetazoline). Many decongestants are recommended to be taken with antihistamines for optimal relief of allergy symptoms. Fluticasone is a steroidal spray also recommended to be taken in conjunction with decongestants. Many of these treatments have well known side effects, including drowsiness (“medicine-head”), or increased blood pressure, and these side effects can be more pronounced when treatments are combined.

Decongestant or steroidal medications for the treatment of allergies may offer an increased benefit to treatment when administered in combination with an MPLA-like compound, as this would in theory address multiple causes of symptoms as a result of allergic responses.

In some embodiments of the invention, the MPLA-like compound is administered prior to, simultaneously, or after decongestant or steroidal allergy treatment.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Example 1

### Preparation of PHAD Micelles in 5% Ethanol.

1 mg of PHAD was wetted for 1 minute in 0.4 mL of 95% ethanol then sonicated for 15 minutes at 40° C. until a clear solution is formed. The solution was removed from the sonication bath and QS to 8 mL with water, resulting in a homogeneous formulation of PHAD micelles 150 nm or smaller in size. The formulation was either used as liquid or lyophilized.

### Example 2

### Preparation of PHAD Powder.

6 mg of PHAD was wetted and dissolved in 3 mL of 95% ethanol at 40° C. and sonicated for 20 minutes at 40° C. until a clear solution of 2 mg/mL PHAD was obtained. 17 mL of water at 40° ° C. was then added and sonicated to fully mix the bulk solution, resulting in a homogeneous formulation of PHAD micelles around 150 nm or smaller in size. 147 mg of HP-β-Cyclodextrin and 147 mg of Trehalose dihydrate were added to the solution under mixing. This solution was then spray dried to obtain a final powder at 2% w/w/w PHAD:HP-β-Cyclodextrin:Trehalose. This particular composition of PHAD was readily soluble in water at concentrations up to 8 mg/mL.

### Example 3

Formulations as prepared in Examples 1 and 2 show robust upregulation of IP-10, as a result of TLR4 stimulation in vitro in mouse macrophages (FIG. 2). IP-10 is an important cytokine associated with stimulation of the TRIF pathway. Evidence of IP-10 upregulation is confirmation that PHAD preparations as noted in this application are capable of selectively stimulating the TRIF pathway, which is further confirmation of the amelioration or reduction of activity mediated through the MyD88 signaling pathway, which supports the amelioration of pro-inflammatory cytokine production for MPLA as a treatment for inflammatory conditions.

### Example 4

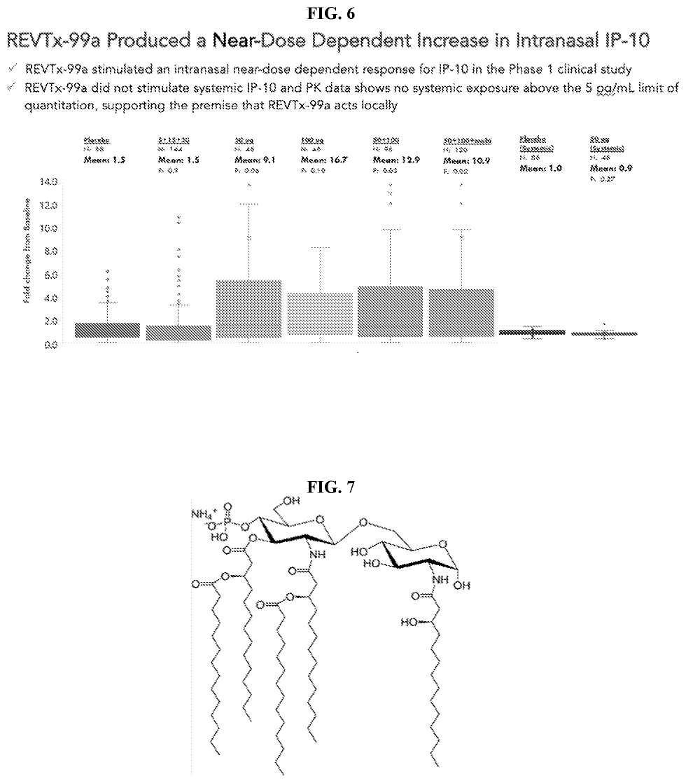

Formulations as prepared in Examples 1 show a dose dependent upregulation of IP-10 when administered intranasally to healthy human volunteers (FIG. 6). Presented is a box and whisker plot of the fold-change values for all data and time-points including the mean fold-change and p-value calculated using a t-test comparing placebo to each data set presented.

### REFERENCES

Alvaro-Lozano M, Akdis C A, Akdis M, et al. EAACI allergen immunotherapy user's guide. Pediatr Allergy Immunol. 2020; 31 Suppl 25(Suppl 25):1-101. doi: 10.1111/pai. 13189.
Bousquet J, Anto J M, Bachert C, et al. Allergic rhinitis. Nat Rev Dis Primers. 2020; 6:95. doi: 10.1038/s41572-00227-0.
Gonzalez-van Horn S, Farrar J D. Interferon at the crossroads of allergy and viral infections. J Leukoc Biol. 2015; 98(2)185-94. doi: 10.1189/jlb.3RU0315-099R.
Loetscher P, Pellegrino A, Gong J H, et al. The ligands of CXC chemokine receptor 3, I-TAC, Mig, and IP10 are natural antagonists for CCR3. J Biol Chem. 2001; 276 (5):2986-91. doi: 10.1074/jbc.M005652200.
Nocera A L, Mueller S K, Stephan J R, et al. Exosome swarms eliminate airway pathogens and provide passive epithelial immunoprotection through nitric oxide. J Allergy Clin Immunol. 2019; 143(4): 1525-1535. doi: 10.1016/j.jaci.2018.08.046

### INCORPORATION BY REFERENCE

All of the U.S. patents, and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A pharmaceutical composition comprising a colloidal formulation of one or more monophosphoryl lipid A (MPLA) like compounds.

2. The pharmaceutical composition of claim 1, wherein the MPLA-like compound is selected from phosphorylated hexaacyl disaccharide (PHAD), PHAD-504, 3D-(6-acyl)-PHAD, 3D-PHAD and any combination thereof.

3. The pharmaceutical composition of claim 2, wherein the MPLA-like compound is PHAD.

4. The pharmaceutical composition of any one of claims 1-3, further comprising a sugar.

5. The pharmaceutical composition of claim 4, wherein the sugar is chosen from a monosaccharide, a disaccharide, a trisaccharide, a linear oligosaccharide, a branched oligosaccharide, a cyclic oligosaccharide, a linear polysaccharide, a branched polysaccharide, or any combination thereof.

6. The pharmaceutical composition of claim 5, wherein the monosaccharide is selected from glucose, dextrose, fructose, galactose, xylose, ribose, and any combination thereof.

7. The pharmaceutical composition of claim 5 or 6, wherein the disaccharide is selected from trehalose, sucrose, maltose, lactose, and any combination thereof.

8. The pharmaceutical composition of any one of claims 5-7, wherein the trisaccharide is selected from nigerotriose, maltotriose, melezitose, maltotriulose, raffinose, kestose. and any combination thereof.

9. The pharmaceutical composition of any one of claims 5-8, wherein the linear or branched oligosaccharide is selected from nigerotetraose, maltotetraose, lychnose, nystose, sesamose, stachyose.

10. The pharmaceutical composition of any one of claims 5 to 9, wherein the cyclic oligosaccharide is selected from alpha-cyclodextrin, beta-cyclodextrin, or gamma-cyclodextrin.

11. The pharmaceutical composition of any one of claims 5-10, wherein the linear or branched polysaccharide is selected from starch, glucan, chitosan, pectin, carboxymethyl cellulose, glycosylaminoglycans, hyaluronic acid, cellulose derivatives, hydroxypropylmethylcellulose (HPMC), dextran, and any combination thereof.

12. The pharmaceutical composition of any one of claims 5-11, wherein the disaccharide is trehalose.

13. The pharmaceutical composition of any one of claims 5-12, wherein the cyclic oligosaccharide is beta-cyclodextrin.

14. The pharmaceutical composition of any one of claims 1-33, wherein the composition further comprises one or more surfactants selected from poloxamer 407, poloxamer 181, dodecyltrimethylammonium bromide (DTAB), n-dodecyl octa (ethylene oxide) (C12E8), n-dodecyl tetra (ethylene oxide) (C12E4), dioctanoyl phosphatidylcholine (C8-lecithin), Polyoxyl 35 castor oil, Cremophor EL (CrEL), Octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), sodium dodecyl sulfate (SDS), and any combination thereof.

15. The pharmaceutical composition of any one of claims 1-14 wherein the composition further comprises a phospholipid selected from phosphatidic acid (“PA”), phosphatidylcholine (“PC”), phosphatidylglycerol (“PG”), phophatidylethanolamine (“PE”), phophatidylinositol (“PI”), phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidylcholine (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), distearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DMPG”), dioleoylphosphatidylglycerol (“DOPG”), dipalmitoylphosphatidylglycerol (“DPPG”), distearoylphosphatidylglycerol (“DSPG”), 1-palmitoyl-2-oleoyl-phosphatidylglycerol (“POPG”), diarachidoylphosphatidylethanolamine (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylethanolamine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPI”), dilauroylphosphatidylinositol (“DLPI”), dilinoleoylphosphatidylinositol, dimyristoylphosphatidylinositol (“DMPI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), di decanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidylserine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinolcoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

16. The pharmaceutical composition of any one of claims 1-15, wherein the pharmaceutical composition is a dry powder.

17. The pharmaceutical composition of any one of claims 1-16, wherein the MPLA-like compound is between 0.5% and 10% by weight of the composition.

18. The pharmaceutical composition of any one of claims 1-17, wherein the sugar is between 80% and 99.5% by weight of the composition.

19. The pharmaceutical composition of claims 14-18, wherein the surfactant or surfactants are between 0.5% and 10% by weight of the composition.

20. The pharmaceutical composition of any one of claims 15-19, wherein the phospholipid or phospholipids are between 0.5% and 10% by weight of the composition.

21. The pharmaceutical composition of any one of claim 20, wherein the pharmaceutical composition is an aqueous composition.

22. The pharmaceutical composition of claim 21, further comprising an organic solvent.

23. The pharmaceutical composition of claim 22, wherein the organic solvent and water are present in a volume to volume ratio of about 1:1500 to about 1:50.

24. The pharmaceutical composition of claim 23, wherein the organic solvent and water are present in a volume to volume ratio of about 1:1000 to about 1:100.

25. The pharmaceutical composition of claim 24, wherein the organic solvent and water are present in a volume to volume ratio of about 1:800.

26. The pharmaceutical composition of any one of claims 22-25, wherein the organic solvent is miscible with water.

27. The pharmaceutical composition of any one of claims 21-26, wherein the organic solvent is selected from an alcohol, glycerin, low molecular weight polyethylene glycol, and low molecular weight poloxamers.

28. The pharmaceutical composition of claim 27, wherein the organic solvent is an alcohol.

29. The pharmaceutical composition of claim 28, wherein the alcohol is selected from methanol, ethanol, isopropanol, or t-butanol.

30. The pharmaceutical composition of claim 29, wherein the alcohol is ethanol.

31. The pharmaceutical composition of any one of claims 21-30, wherein the pharmaceutical composition has an MPLA-like compound(s) concentration of about 1 μg/mL to about 30 mg/mL.

32. The pharmaceutical composition of any one of claims 21-30, wherein the pharmaceutical composition has a MPLA-like compound(s) concentration of about 50 μg/mL to about 500 μg/mL.

33. The pharmaceutical composition of any one of claims 21-30, wherein the pharmaceutical composition has a MPLA-like compound(s) concentration of about 125 μg/mL.

34. The pharmaceutical composition of any one of claims 21-30, wherein the pharmaceutical composition has a MPLA concentration of about 250 g/mL.

35. The pharmaceutical composition of any one of claims 21-30, wherein the pharmaceutical composition has a MPLA-like compound(s) concentration of about 250 μg/mL.

36. The pharmaceutical composition of any one of claims 1-35, further comprising a stabilizer.

37. The pharmaceutical composition of any one of claims 1-36, wherein the composition comprises micelles having an average diameter or length of about 1 nm to about 1000 nm.

38. The pharmaceutical composition of claim 37, wherein the composition comprises micelles having an average diameter or length of about 50 nm to about 500 nm.

39. The pharmaceutical composition of claim 38, wherein the composition comprises micelles having an average diameter or length of about 100 nm to about 500 nm.

40. The pharmaceutical composition of any one of claims 1-39, further comprising a mucoadhesive agent.

41. The pharmaceutical composition of claim 40, wherein the mucoadhesive agent is selected from is selected from cellulose derivatives, polyacrylates, a starch, chitosan, glycosylaminoglycans, hyaluronic acid, cellulose derivatives, polyacrylates, and any combination thereof.

42. The pharmaceutical composition of any one of claims 1-41, further comprising a pH modifier, a pH buffer, an emulsifier, a tonicity modifier, a stabilizer, a preservative, a surfactant, a bulking agent, a flavorant, or any combination thereof.

43. The pharmaceutical composition of claim 42, wherein the bulking agent is selected from mannitol, trehalose, chitosan, hydroxypropylmethylcellulose (HPMC), dextran, pea starch, and sucrose.

44. The pharmaceutical composition of any one of claims 1-43, wherein the composition comprises micelles.

45. The pharmaceutical composition of any one of claims 1-44, wherein the composition comprises liposomes.

46. The pharmaceutical composition of any one of claims 1-45, wherein the composition comprises nanoparticles.

47. The pharmaceutical composition of any one of claims 1-46, wherein the composition comprises microparticles.

48. A method of preparing a pharmaceutical composition of any one of claims 1-47, comprising:
a. dissolving one or more MPLAs in an organic solvent to form an organic solvent/MPLA solution;
b. combining the organic solvent/MPLA solution with water to form a colloidal suspension comprising the one or more MPLAs.

49. The method of claim 48, wherein step (a) or (b) is performed under sonication.

50. The method of claim 48 or 49, wherein the organic solvent and the water are present in a volume to volume ratio of about 1:1500 to about 1:50.

51. The method of any one of claims 48-50, wherein the organic solvent and water are present in a volume to volume ratio of about 1:1000 to about 1:100.

52. The method of claim 51, wherein the organic solvent and water are present in a volume to volume ratio of about 1:800.

53. The method of any one of claims 48-52, wherein one or more surfactants are added in step (a) or (b).

54. The method of claim 53, wherein the surfactant is carboxymethyl cellulose.

55. The method of any one of claims 48-54, wherein one or more phospholipids is added in step (a) or (b).

56. The method of any one of claims 48-55, wherein the mixture in step (b) is lyophilized.

57. The method of any one of claims 48-56, wherein the mixture in step (b) is spray dried.

58. The method of any one of claims 48-57, wherein the mixture in step (b) is stabilized with trehalose.

59. The method of any one of claims 48-58, wherein step (a) or step (b) is performed at an elevated temperature.

60. The method of claim 59, wherein the elevated temperature is about 30° C. to about 50° C.

61. The method of claim 60, wherein the elevated temperature is about 40° C.

62. The method of any one of claims 48-61, wherein the mixture in step (b) has MPLA concentration of about 125 μg/mL.

63. The method of any one of claims 48-62, wherein the mixture in step (b) has a MPLA concentration of ranging from about 1 g/mL to about 1000 μg/mL; about 20 μg/mL to about 500 g/mL, about 100 μg/mL to about 300 g/mL, about 250 μg/mL, or about 125 μg/mL.
